(19) United States
(12) Patent Application Publication
(10) Pub. No.: US 2024/0353408 A1
(43) Pub. Date: Oct. 24, 2024

(54) MXENE-GRAPHENE FIELD EFFECT TRANSISTOR VIRUS SENSOR

(71) Applicant: The Curators of the University of Missouri, Columbia, MO (US)

(72) Inventors: Chenglin Wu, Rolla, MO (US); Yue-Wern Huang, Rolla, MO (US); DongHyun Kim, Rolla, MO (US); Yang Wang, Rolla, MO (US); Yanxiao Li, Rolla, MO (US); Risheng Wang, Rolla, MO (US); Jiaoli Li, Rolla, MO (US); Yuwei Zhang, Rolla, MO (US)

(73) Assignee: The Curators of the University of Missouri, Columbia, MO (US)

(21) Appl. No.: 18/684,297

(22) PCT Filed: Aug. 25, 2022
(86) PCT No.: PCT/US2022/041497

§ 371 Date: Feb. 16, 2024

Related U.S. Application Data
(60) Provisional application No. 63/238,454, filed on Aug. 30, 2021.

(51) Int. Cl. G01N 33/569; G01N 27/414; G01N 33/543

(52) U.S. Cl. CPC G01N 33/56983; G01N 27/4145; G01N 33/5438; G01N 2333/11; G01N 2333/165; G01N 2469/10

(57) ABSTRACT

A sensor for detecting virus, including virus particles and/or genetic sequences, and method of fabrication. The sensor includes a field effect transistor (FET) having source and drain electrodes formed on a substrate and a two-dimensional virus sensing transduction material (VSTM) film formed on the FET. The VSTM film is configured to collect a sample collected from a subject and comprises MXene-graphene. The VSTM film has an antibody corresponding to the virus particles to be detected linked to the film. A drain-source current response of the FET is representative of an amount of the virus particles in the sample for indicating an infection.

### CROSS-REFERENCE TO RELATED APPLICATION

This application claims priority from U.S. Provisional Patent Application No. 63/238,454, filed Aug. 30, 2021, the entire disclosure of which is incorporated herein by reference.

### BACKGROUND

Influenza viruses and coronaviruses are among the most common causes of infectious viral respiratory diseases. Point-of-care testing becomes critical in real-time detection and tracing of viral diseases, such as severe acute respiratory syndrome coronavirus 2 (SARS-COV-2). Real-time sensing of these viruses has attracted a significant amount of research interests and various sensing mechanisms have been explored. Among them, electro-chemical immuno-sensing has a fast response time and relatively easy signal transduction pathway for data interpretation. However, real-time detection and understandable data output requires a highly sensitive virus sensing transduction material (VSTM) that can selectively sense the virus and transduce the sensing signal into electronic signals. While two-dimensional (2D) materials, including graphene, are uniquely positioned as a VSTM for bio-sensing, further improvement of the chemical reactivity of graphene-like VSTMs is needed to achieve higher selectivity and sensitivity for rapid and real-time bio-sensing.

Recent studies have also shown success using graphene field effect transistor (FET) sensing for influenza and SARS-COV-2 virus. However, due to the relative low signal-to-noise ratio of the graphene sensing material, the device lacks robustness and requires pre-processing of the virus sample. Therefore, a VSTM that has higher sensitivity and signal-to-noise ratio is needed to improve the robustness and allows minimum sample preparation, which enables the automatic and real-time sensing of influenza and SARS-COV-2 viruses.

### SUMMARY

Aspects of the present disclosure relate to a 2D transition metal carbide (MXene) on graphene structure to combine the high chemical reactivity of MXene and continuity of graphene for forming an ultrasensitive virus sensing transduction material (VSTM). The integration of ultrasensitive biological sensing with digital devices, such as touchscreen mobile phones and laptops, further substantially improve the efficiency of point-of-care testing. This integration allows swift tracking of airborne viral load as well as easy access to biological sensing information using the touchscreen. Advantageously, aspects of the present disclosure provide a highly sensitive VSTM that can selectively sense the virus and transduce the sensing signal into electronic signals in order to be transmitted to touchscreen devices.

In an aspect, a sensor for detecting virus particles includes a field effect transistor (FET) having source and drain electrodes formed on a substrate and a two-dimensional virus sensing transduction material (VSTM) film formed on the FET. The VSTM film is configured to collect a sample collected from a subject and comprises MXene-graphene. The VSTM film has a probe, such as an antibody or deoxyribonucleic acid (DNA), corresponding to the virus particles to be detected linked to the film. A drain-source current response of the FET is representative of an amount of the virus particles in the sample for indicating an infection.

In another aspect, a method of fabricating a virus sensor includes patterning a source electrode and a drain electrode of a FET on a substrate, growing a monolayer graphene by chemical vapor deposition (CVD) and depositing the graphene on the substrate, and depositing a layer of MXene on the graphene to form a continuous virus sensing transduction material (VSTM) film formed on the FET. The VSTM film comprises MXene-graphene and is configured to receive a sample from a subject for detecting particles of a virus. The method also includes linking a probe, such as an antibody or DNA, corresponding to the virus to be detected to the VSTM film. A drain-source current response of the FET is representative of an amount of particles of the particles of the virus in the sample for indicating an infection.

In yet another aspect, a face mask comprises a substrate configured to cover a subject's mouth and nose when worn by the subject and a sensor located on an inner surface of the substrate. The sensor is configured to detect an infection from exhaled breath of the subject when the substrate is worn by the subject. The sensor comprises FET having source and drain electrodes formed on the substrate. The sensor also includes a two-dimensional VSTM film formed on the FET. The VSTM film is configured to collect a sample of the exhaled breath of the subject and comprises MXene-graphene having a probe, such as an antibody or DNA, corresponding to the virus particles to be detected linked to the film. A drain-source current response of the FET is representative of an amount of the virus particles in the sample for indicating an infection.

Other objects and features of the present disclosure will be in part apparent and in part pointed out herein.

### BRIEF DESCRIPTION OF THE DRAWINGS

FIG. 1 illustrates a MXene-graphene field effect transistor (FET) sensor according to an embodiment.

FIG. 2 illustrates an example process for fabricating the MXene-graphene FET sensor of FIG. 1.

FIGS. 3A-3C illustrate example Raman spectra of graphene and MXenes according to an embodiment.

FIG. 3D illustrates example Fourier-transform infrared (FTIR) spectroscopy plots for MXenes according to an embodiment.

FIG. 3E illustrates atomic force microscopy (AFM) images for MXenes according to an embodiment.

FIGS. 4A to 4D provide example data for calibrating the sensor of FIG. 1.

FIGS. 5A and 5B the sensor of FIG. 1 configured for sensing the SARS-COV-2 spike antibody for recombinant 2019-nCOV spike protein and for sensing the influenza A (H1N1) HA polyclonal antibody for inactivated influenza A (H1N1) virus.

FIGS. 5C to 5H provide example data for calibrating the sensor of FIG. 1 for specificity verification.

FIG. 6 Illustrates a real-time resistance monitor system including the sensor of FIG. 1.

FIG. 7A illustrate an auto-balancing bridge impedance measurement circuit and a voltage divider resistance measurement circuit, respectively, for use with the system of FIG. 6.

FIGS. 8A to 8F illustrate example curves obtained using a variety of experimental setups.

FIG. 9 is a photo of the MXene-graphene FET sensor of FIG. 1 on a face mask and an enlarged scanning electron microscope (SEM) image of MXene (Ti₂CTₓ) flakes on graphene.

FIG. 10 illustrates a sensor functionalization process for nucleic acid detection according to another embodiment.

FIG. 11 illustrates a MXene-graphene FET sensor for use with the process of FIG. 10.

FIG. 12 provides example data for calibrating the sensor of FIG. 11.

FIG. 13 illustrates fully matching and mismatching probe deoxyribonucleic acid (DNA) and target DNA as detected using the sensor of FIG. 11.

FIGS. 14A and 14B compare electrical double layer (EDL) structures of graphene and MXene-graphene, respectively, according to an embodiment.

FIGS. 15A and 15B compare bandgap structures of graphene and MXene-graphene, respectively, according to an embodiment.

Corresponding reference numbers indicate corresponding parts throughout the drawings.

### DETAILED DESCRIPTION

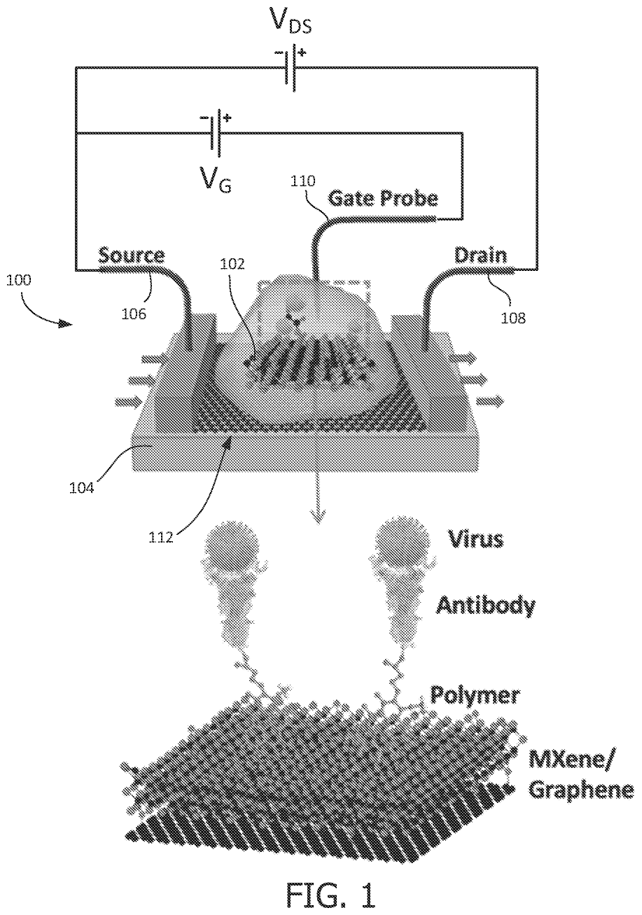

Referring now to the drawings, FIG. 1 illustrates a field effect transistor (FET) sensor 100 embodying aspects of the present disclosure. In an embodiment, the sensor 100 is configured for sensing influenza and/or SARS-COV-2. The sensor 100 includes an ultra-sensitive virus sensing transduction material (VSTM) 102 on a substrate 104, a source electrode 106, a drain electrode 108, and a gate 110. The sensor 100 combines the high chemical sensitivity of 2D transition metal carbide (MXene) and the continuity of large-area high quality graphene to form the VSTM 102. Through polymer linking, sensor 100 utilizes antibody-antigen binding to achieve electrochemical signal transduction when viruses are deposited onto the surface of VSTM 102. In an embodiment, the MXene-graphene VSTM 102 is configured to directly receive viruses. In an embodiment, sensor 100 is packaged in a silicone such as polydimethylsiloxane (PDMS) that includes a microfluid receiver 112, such as a channel or chamber, for collecting a sample for testing.

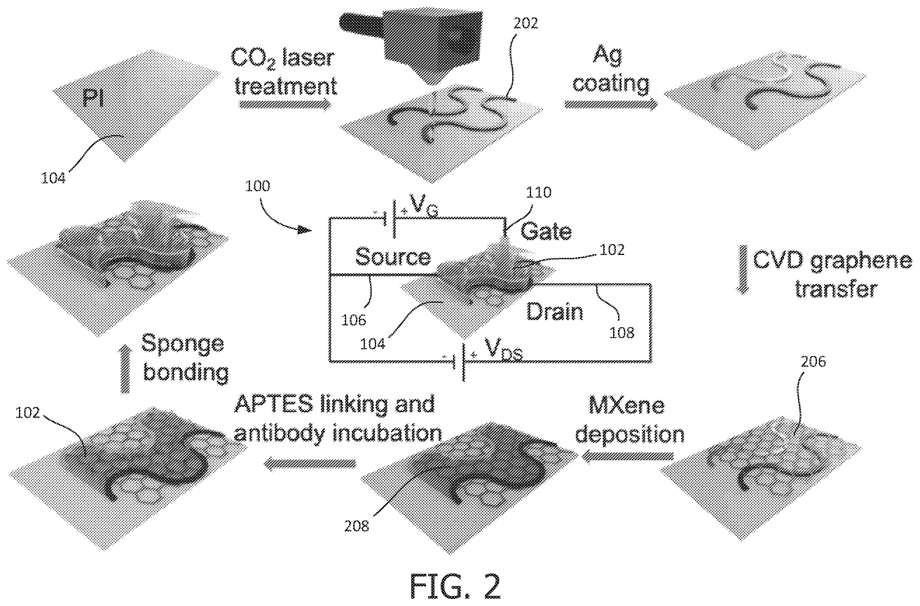

FIG. 2 illustrates an example process for fabricating MXene-graphene FET sensor 100 in which a carbon dioxide (CO₂) laser patterns electrodes 202 on substrate 104, for example, a serpentine pattern. A silver (Ag) coating process provides a conductive Ag coating on the electrodes 202. According to an embodiment, a thin sintering aid layer consisting of polyvinyl alcohol (PVA) paste and calcium carbonate (CaCO3) nanoadditives in water are first applied at a target sensor location on substrate 104 for the subsequent sensor integration. After confirming the reduced surface roughness of substrate 104, Ag nanoparticle ink is directly printed on the sintering aid layer. Drying forms Ag electrodes 202 with patterned conductive traces for external connection.

Next, the MXene-graphene VSTM 102 is deposited on substrate 104. A relatively large-area, monolayer graphene layer 206 is grown by chemical vapor deposition (CVD) and deposited onto substrate 104 using, for example, a wet transfer method. A MXene layer 208 is subsequently deposited onto the graphene 206 using, for example, an interfacial deposition approach. Suitable MXenes includes titanium carbide (Ti₂C or Ti₂CTₓ) and titanium aluminum carbide (Ti₂AlC) The MXene-graphene structure comprises VSTM 102. This MXene-graphene hybrid permits forming a covalently bonded polymer probe instead of noncovalently bonded as in graphene. In addition, the hybrid MXene-graphene is a continuous film, which provides high signal-to-noise ratio.

In an embodiment, sensor 100 includes an antibody-antigen sensing mechanism on the graphene and MXene layers 206, 208. The surface of the MXene-graphene VSTM 102 is functionalized using an aminosilane such as (3-Aminopropyl) triethoxysilane (APTES) and linking the corresponding influenza A (H1N1) antibody or SARS-COV-2 spike antibody to the APTES. When inactivated influenza A (H1N1) virus and recombinant 2019-nCOV spike protein bind with their corresponding antibodies, the associated change of surface charge can be measured with the circuit of sensor 100 shown in FIG. 1 and reflected from the drain-source current-voltage response.

Among various sensing mechanisms, the electrochemical immuno-sensing has the fastest response time and the easiest signal transduction pathway. In this sensing mechanism, antibodies specific to the virus alter surface charge distribution of VSTM 102 after virus binding. The changed surface signal can then propagate to various electrochemical signals. The electrochemical signal transduction is commonly achieved using the FET for amplified signals. Unlike a graphene-based FET, which has a relatively low signal-to-noise ratio (SNR), the present MXene-graphene sensor 100 provides high sensitivity and SNR.

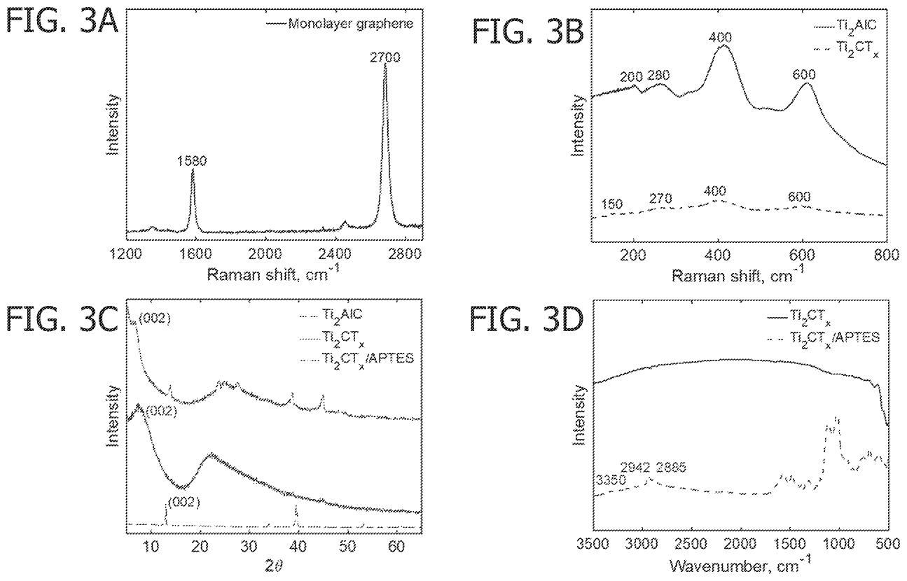

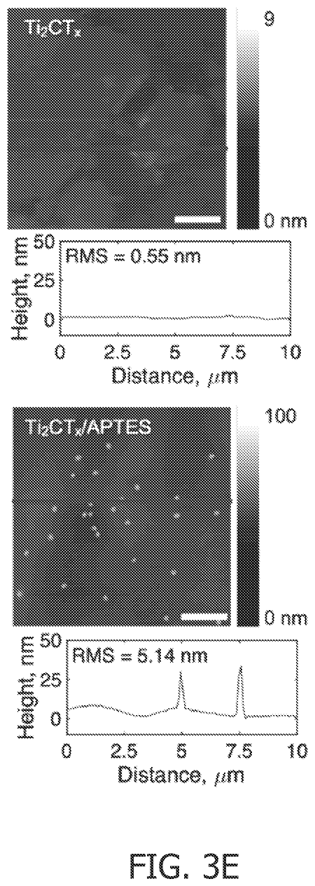

Referring now to FIGS. 3A to 3E, material characteristics of sensor 100 are shown. Characterization of the MXene-graphene VSTM 102 is characterized using Raman spectrometry, Fourier-transform infrared spectroscopy (FTIR), X-ray diffraction (XRD), and atomic force microscopy (AFM).

FIG. 3A illustrates an example Raman spectrum of monolayer graphene 206 featuring Raman peaks at 1580 cm⁻¹ and 2683 cm⁻¹. FIG. 3B illustrates example Raman spectra of MXenes 208 Ti₂AlC and Ti₂CTₓ. In FIG. 3B, characteristic Raman peaks of MXene (Ti₂AlC) at 200, 280, 400, and 600 cm⁻¹, compared with 400 and 600 cm⁻¹ for the MAX precursor (Ti₂CTₓ) as shown indicates the successful removal of Al from Ti₂AlC. FIG. 3C illustrates example XRD plots for Ti₂AlC, Ti₂CTₓ, and APTES functionalized Ti₂CTₓ. According to an embodiment, FTIR, XRD, and AFM scans are used to confirm that the MXene surface is chemically functionalized with APTES. FIG. 3D illustrates example FTIR plots for Ti₂CTₓ and APTES functionalized Ti₂CTₓ. The FTIR spectrum of APTES-MXene (i.e., VSTM 102) of FIG. 3D shows several new vibration peaks in comparison with that of MXene 208. For instance, new peaks at 1500 cm⁻¹ and 3350 cm⁻¹ (the stretching vibration mode of the free amine from APTES) appears in the FTIR spectrum of Ti₂CTₓ/APTES. This indicates the successful covalent coupling of APTES to Ti₂CTₓ MXene. Since MXene 208 has two-dimensional (2D) structure, the change of interlayer distance after modification of silanes is also examined. The 002 peak of Ti₂CTₓ/APTES in the XRD pattern of FIG. 3C is shifted towards a smaller angle compared with that of Ti₂CTₓ MXene (from 7.70° to 6.56°), resulting in an increase of d-spacing to 1.35 nm. This may be attributed to the intercalation effect of APTES.

FIG. 3E displays AFM images for Ti₂CTₓ and APTES functionalized Ti₂CTₓ. Scale bar is 500 nm. APTES is also observed from the AFM images (bright spots in FIG. 3E). After APTES modification, the surface roughness (RMS) of MXene 208 increases from 0.521 nm to 4.454 nm in the illustrated example. In these contexts, APTES is successfully functionalized on the surface of MXene 208, which acts as a protein linker to immobilize antibodies to the MXene surface.

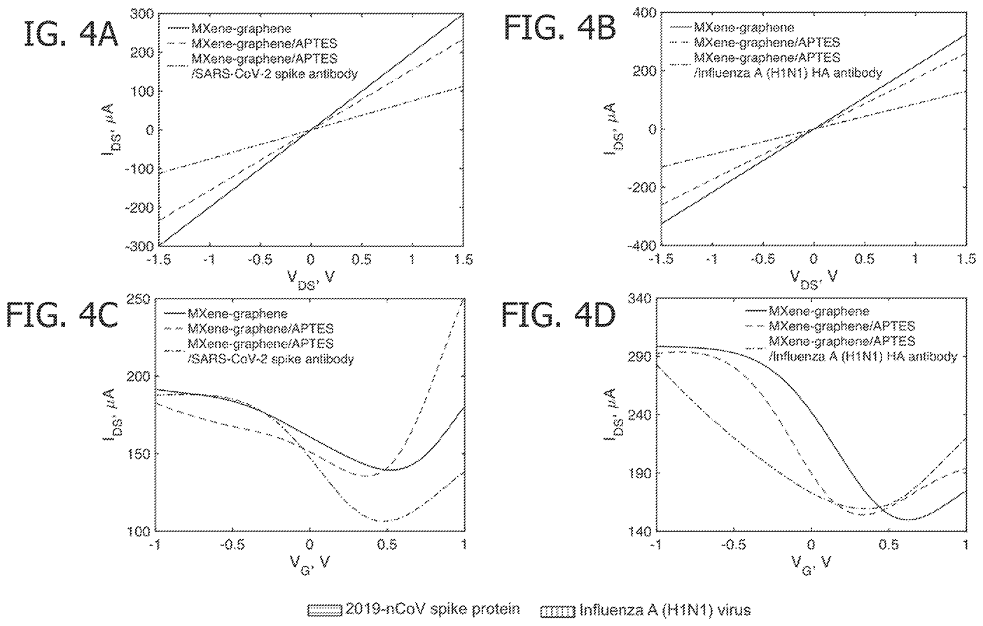

FIGS. 4A to 4D provide example data for calibrating sensor 100 through electrochemical signal testing. FIGS. 4A and 4C illustrate electrical characterizations of pristine immobilized MXene-graphene 208-206, APTES functionalized MXene-graphene of VSTM 102, and APTES functionalized MXene-graphene with the SARS-COV-2 spike antibody. Similarly, FIGS. 4B and 4D illustrate electrical characterizations of pristine immobilized MXene-graphene 208-206, APTES functionalized MXene-graphene, and APTES functionalized MXene-graphene of VSTM 102 with the influenza A (H1N1) HA polyclonal antibody. In this regard, FIGS. 4A and 4B show I_{DS}-V_{DS} (V_{G}=0) curves of each step of the material treatment of sensor 100, and FIGS. 4C and 4D show I_{DS}-V_{G} (V_{DS}=0.5 V) curves of each step of the material treatment of sensor 100.

The I_{DS}-V_{DS} curves of FIGS. 4A and 4B for the FET sensor 100 after APTES functionalization and immobilization of the antibody onto VSTM 102 indicate the presence of the SARS-COV-2 spike antibody or influenza A (H1N1) antibody on VSTM 102. Over a range from −1.5 to +1.5 V of V_{DS} before and after attachment of APTES and the antibody, the slopes (dI_{DS}/dV_{DS}) decreased. These differences in slopes indicate the successful introduction of APTES, influenza A (H1N1) antibody or SARS-COV-2 spike antibody. In addition, as shown in FIGS. 4C and 4D, after APTES functionalization, an obvious negative shift is observed in I_{DS}-V_{G} curves due to the n-doping effect of the amine group. However, the transfer curve is shifted positively after immobilization of the antibody due to negative charge of the antibody, which exerted a p-doping effect on VSTM 102.

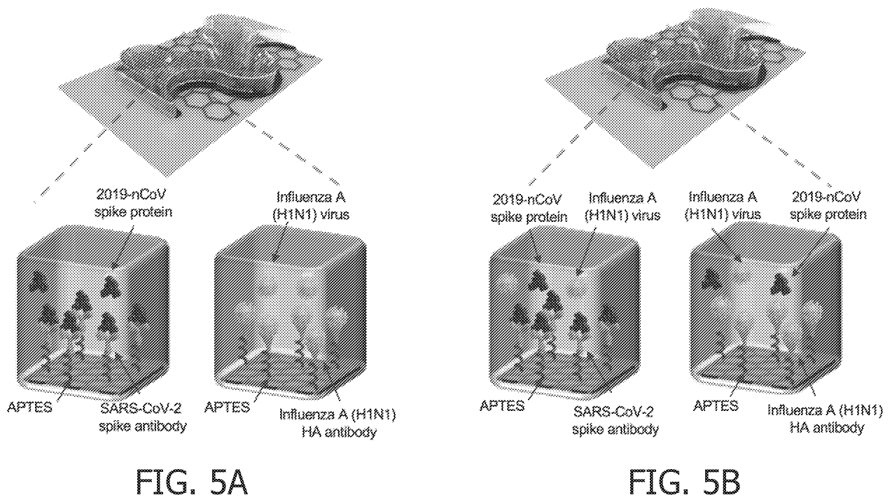

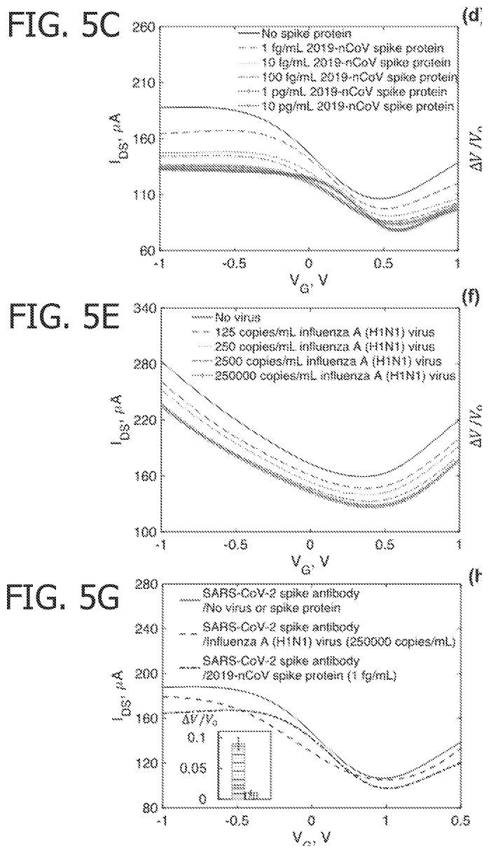

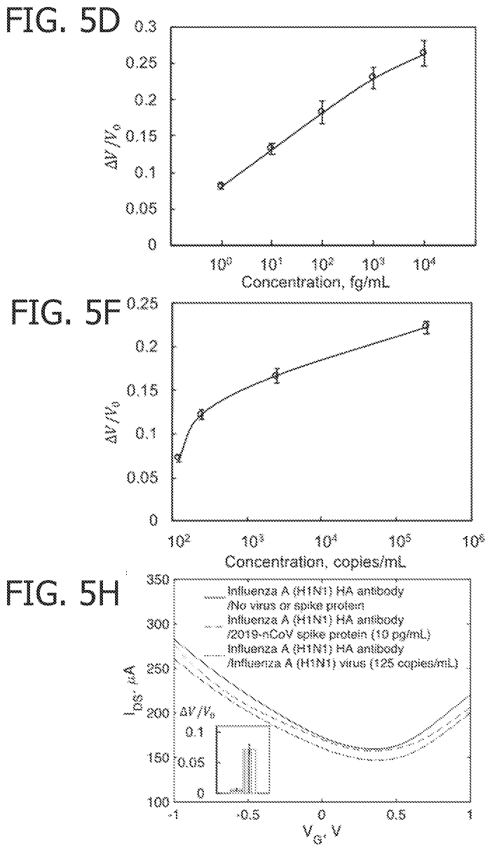

FIG. 5A illustrates immobilized sensor 100 configured for sensing the SARS-COV-2 spike antibody for recombinant 2019-nCOV spike protein and for sensing the influenza A (H1N1) HA polyclonal antibody for inactivated influenza A (H1N1) virus. FIG. 5B illustrates sensing specificity verification for differentiating the antibodies.

FIGS. 5C to 5H provide example data for calibrating sensor 100 for specificity verification. In FIG. 5C, I_{DS}-V_{G} curves (V_{DS}=0.5 V) are shown for applying various concentrations of recombinant 2019-nCOV spike protein to the SARS-COV-2 spike antibody immobilized sensor 100. FIG. 5D shows a corresponding

$$\frac{Δ V}{{V}_{0}}$$

versus concentration curve. FIG. 5E shows I_{DS}-V_{G} curves (V_{DS}=0.5 V) for applying various concentrations of inactivated influenza A (H1N1) virus to the influenza A (H1N1) HA polyclonal antibody immobilized sensor 100 and FIG. 5F shows a corresponding

$$\frac{Δ V}{{V}_{0}}$$

versus concentration curve (x axis is log plotted). FIGS. 5G and 5H illustrate I_{DS}-V_{G} curves (V_{DS}=0.5 V) for the sensing specificity study, with

$$\frac{Δ V}{{V}_{0}}$$

values in subset figures. The errors are standard deviations.

As described above, point-of-care testing becomes critical in real-time detection and tracing of the severe acute respiratory syndrome coronavirus 2 (SARS-COV-2) and the like. The integration of ultrasensitive biological sensing with digital devices, such as touchscreen mobile phones and laptops, can further substantially improve the efficiency of point-of-care testing. This integration allows swift tracking of airborne viral load as well as easy access to biological sensing information using the touchscreen. However, such an approach desires a highly sensitive virus sensing transduction material (VSTM 102) that can selectively sense the virus and transduce the sensing signal into electronic signals in order to be transmitted to touchscreen devices. Because two-dimensional (2D) materials including graphene are essentially all surfaces, they are uniquely positioned as one of the best VSTM for bio-sensing.

To this end, aspects of the present disclosure take advantage of the high chemical sensitivity of hybrid MXene-graphene 2D materials in combination with microfluidic devices and electro-chemical sensing to develop a touchscreen-based device to detect and trace airborne viral pathogens like SARS-COV-2 in a real time mode.

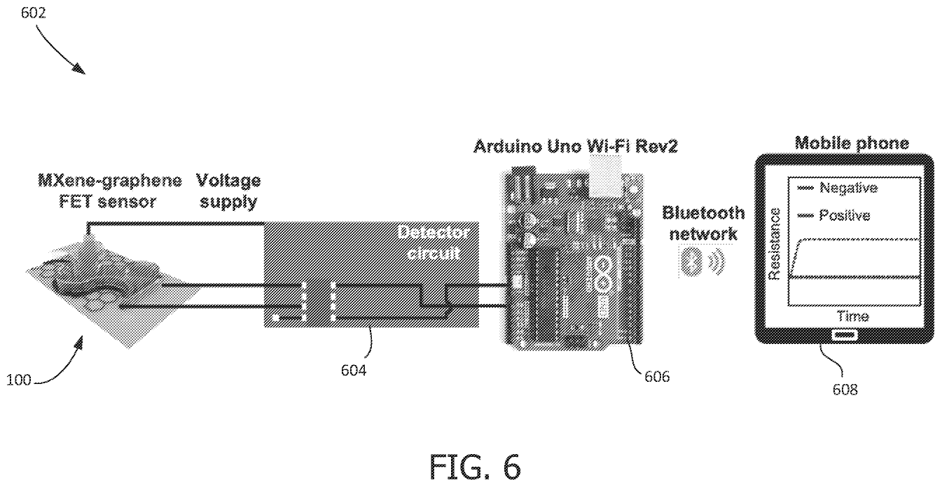

FIG. 6 illustrates a real-time resistance monitor system 602 utilizing sensor 100 for bio-sensing. In operation, a patient exposes the VSTM 102 of sensor 100 to a sample for testing. An integrated influenza/SARS-COV-2 biosensor 100 in the illustrated embodiment is configured to receive a bioaerosol sample from, for example, a patient's breath or droplets emanated from oral cavities to rapidly detect influenza and SARS-COV-2 viruses. In an alternative embodiment, sensor 100 is coupled to a touch-based bio-sample collector for receiving the sample for testing. A dielectrophoresis (DEP) sample concentrator may be used for concentrating the sample before exposing it to the VSTM 102 of sensor 100.

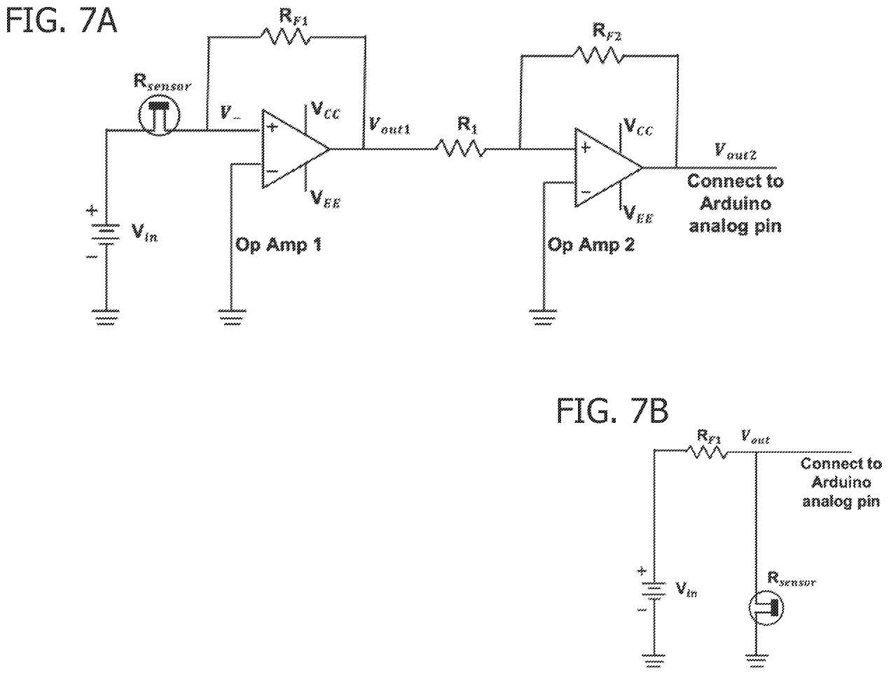

As shown in FIG. 6, the monitor system 602 includes a detector circuit 604 coupled to sensor 100. The detector circuit 604 measures I_{DS}-V_{DS} signals of FET sensor 100. The FET sensor 100 signals change with functionalization and the normalized current versus injection of the virus varies at different concentrations with time. In an embodiment, detector circuit 604 comprises an auto-balancing bridge impedance measurement circuit 702 and a voltage divider resistance measurement circuit 704, as shown in FIGS. 7A and 7B, respectively.

Referring again to FIG. 6, an input/output (I/O) circuit 606 coupled to the detector circuit 604 communicates the detected sensor signals to a computing device 608 (e.g., a smartphone) for processing and display to a user. In the illustrated embodiment, the I/O circuit 606 is configured for communicating the sensor signals to the computing device 608 wirelessly. Alternatively or additionally, I/O circuit 606 comprises a visual indicator, such as an LED light, for indicating presence of the virus in the sample.

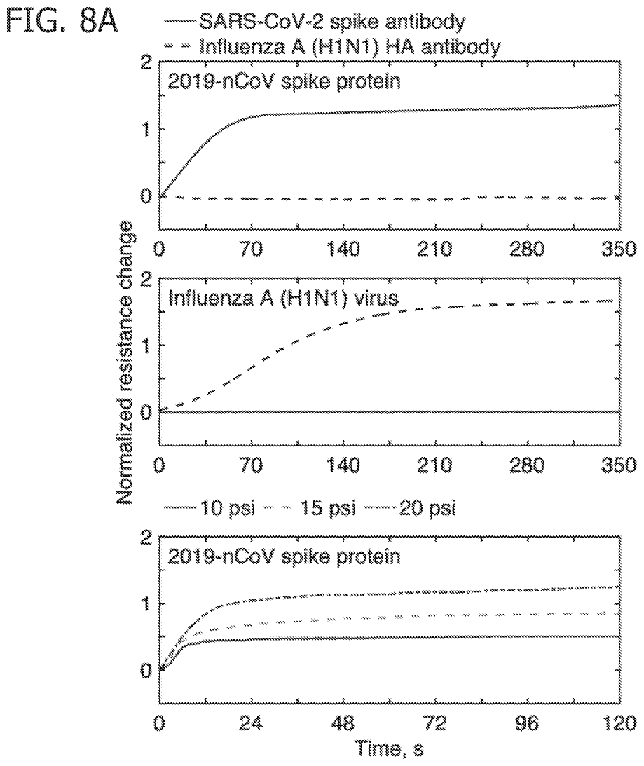

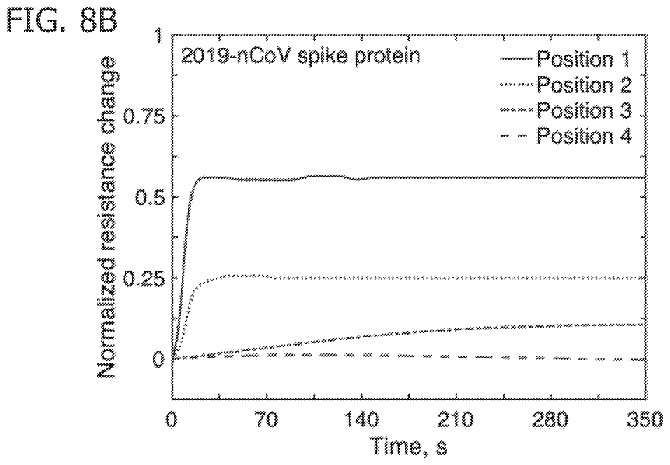

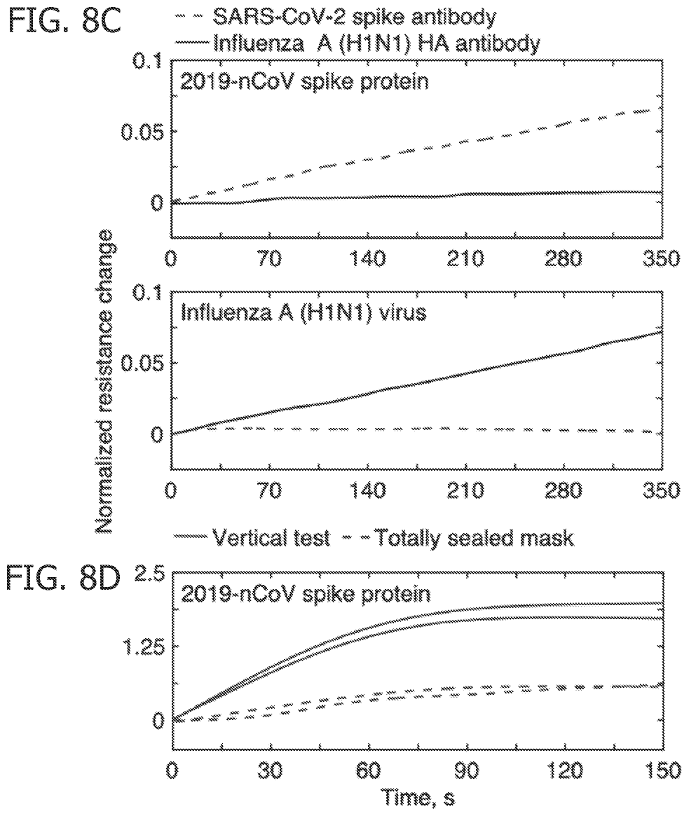

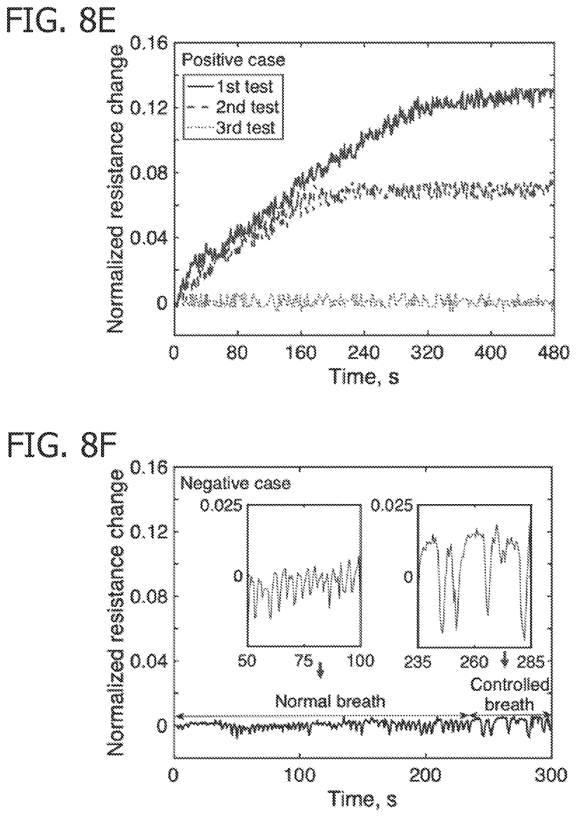

FIGS. 8A to 8F illustrate example curves obtained using a variety of experimental setups. FIG. 8A shows normalized resistance change versus time curves for an experimental setup of sensing specificity verification and flow rate effect in aerosol experiments. FIG. 8B shows normalized resistance change versus time curves for an experimental setup of viral load study in aerosol experiments. FIG. 8C shows normalized resistance change versus time curves of a sensing specificity study. FIG. 8D shows normalized resistance change versus time curves for an experimental setup in which sensor 100 is totally sealed compared to being vertically placed. FIG. 8E shows normalized resistance change versus time curves for real-human on-mask sensing and real-time monitoring of a single patient positive case. FIG. 8F shows normalized resistance change versus time curves for real-human on-mask sensing and real-time monitoring of a single patient negative case (sublets are zoom in curves under normal breath and controlled breath}.

In an embodiment, aspects of the present disclosure enable rapid and automatic detection and tracing influenza and SARS-COV-2 viruses and can be utilized as fast screening for airport and public transit stations and/or utilized by first-line responders during the COVID-19 pandemic.

Moreover, aspects of the present disclosure are unique in terms of high sensitivity and selectivity in viruses sensing. Very small amount of sample (2-3 ml) can be detected with low limit of detection (LOD) about 250 copies/ml of virus concentration. In an embodiment, a breath sample collector can collect the bioaerosol samples directly from breath or water droplets emanated from oral cavity.

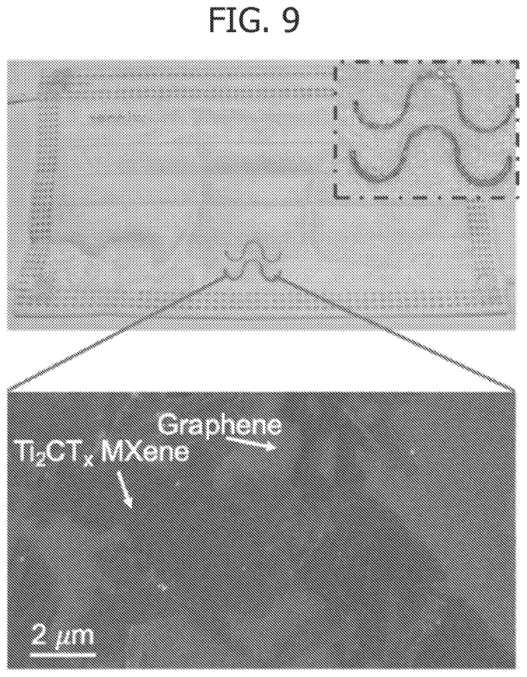

In an embodiment, a smart face mask, such as shown in FIG. 9, is integrated with a flexible 2D material virus sensor for conveniently and effectively detecting SARS-COV-2 viruses emanated from respiratory activities. FIG. 9 is a photo of the MXene-graphene FET sensor of FIG. 1 on a face mask and an enlarged scanning electron microscope (SEM) image of MXene (Ti₂CTₓ) flakes on graphene.

Personal protection equipment, including face masks and face shields, are the ideal candidates to be installed with sensors for noninvasive testing and monitoring SARS-COV-2 viruses from respiratory activities. Although the detection of SARS-COV-2 viruses in the form of bioaerosols (aerosols that carry the virus) from respiratory cavities is challenging, the cumulative collection over time makes the monitoring much easier. In an embodiment of the virus sensor, the hybrid MXene-graphene 2D materials 208, 206 are used as the virus sensing transduction material (VSTM) 102 with high surface virus linker density. Next, according to aspects of the present disclosure, monoclonal antibodies are linked on the hybrid MXene-graphene VSTM 102 to prepare the FET sensor 100 for SARS-COV-2 virus detection (in terms of spike protein) with high sensitivity, selectivity, and low limit of detection (e.g., 1 fg/ml). Because of the potential mechanical deformations on the face mask, the virus sensor 100 is designed to be flexible to withstand the various motions exerted to the face mask during daily use (e.g., bending, twisting, and stretching). In addition, the flexible virus sensor 100 is directly fabricated on the face masks with a scalable and low-cost fabrication approach.

Advantageously, a Band-Aid-size sensor 100 is easily attached to the face mask or shield to collect the accumulated SARS-COV-2 viruses through direct breathing. Consequentially, this removes the inconvenient testing routine and provides warnings on a possible breach of the protection and informs early infections. Importantly, such a sensor can shift the current practice paradigms of detection and monitoring of the infectious conditions of our first responders and medical staff, and even the general public, to protect our most precious medical resources.

Advantageously, the MXene-graphene hybrid VSTM 102 of the present disclosure has high sensitivity, selectivity, and signal-to-noise ratio in terms of SARS-COV-2 sensing. Moreover, implementing the MXene-graphene FET sensor 100 on a low cost, flexible system permits integration with a face mask or the like to achieve real-time detection and monitoring. Compared to the existing real-time reverse transcription-polymerase chain reaction (RT-PCR) approach, the smart face mask is more effective for SARS-COV-2 sensing due to its capabilities for high sensitivity/selectivity, fast response (˜30 milliseconds, in contrast to 30 minutes for RT-PCR), and convenient sample collection from respiratory activities. The smart face mask technology can also be applied to other respiratory infectious diseases.

Referring further to fabrication of sensor 100, in an embodiment, the chemical structure of the sintering aid layer is characterized by the FTIR and nuclear magnetic resonance (HNMR and CNMR). The surface functionality is determined by attenuated total reflection (ATR)-FTIR over a wavelength range of 400-3100 cm⁻¹. The thermal behavior of the sintering aid layer is characterized using differential scanning calorimetry (DSC) and thermogravimetric analysis (TGA). Tensile and compressive mechanical properties are tested on an Instron mechanical tester. Specifically, the stress-strain curves of the sintering aid layer are obtained to inform their mechanical properties.

In an embodiment, soft microfluidics are created for sample filtering and collection. For example, a microfluidic channel (500 μm for both the width and height) and a gate chamber (4×24 mm in the width and length) are first patterned on a thin PDMS film having a thickness of 600 μm. Next, a porous filter is prepared on the other end of the soft microfluidics. The fabrication process starts with the cutting of a cavity in a PDMS slab by a CO₂ laser. After applying a layer of salt crystals at the bottom of the cavity to half of the cavity thickness, Ecoflex diluted with heptane (w/w=1:1) is poured into the cavity to just immerse the salt layer. Once the Ecoflex is cured, the salt crystals in the cavity are dissolved by distilled water, followed by drying to leave a porous layer at the bottom. After flipping over the cavity with the porous Ecoflex on top, it is ready for integration with the FET sensor 100.

Aspects of the present disclosure achieve the following features for daily monitoring of SARS-COV-2:1) easy detection of SARS-COV-2 virus from respiratory activities with the face mask; 2) quantitative assessment of COVID onset and progression with continuous and cumulative monitoring of SARS-COV-2; and 3) low-cost and point-of-care design. The users include, but are not limited to, both medical and non-medical front-line responders. The daily monitoring capability of the smart face mask can also provide clinically relevant data for prevention and intervention in the general public population. Smart face mask technology can also be applied to other respiratory infectious diseases.

From the system level, aspects of the present disclosure combine the electrostatic sample collection, concentration, and sensing on an integrated micro fluid chip, which has not been seen in the known art. This combination allows the collection of samples directly from a patient's breath and permits specific detection with ultrasensitivity.

For the FET sensing, the hybrid MXene-graphene sensing material is more sensitive to the virus per sensing area than other materials (including graphene). A sensor embodying aspects of the present disclosure is unique in that it can selectively sense influenza or SARS-CoV-2 virus using breathing in comparison with existing devices that require combinations of signals to verify the virus.

For example, a device embodying aspects of the present disclosure exhibits the following characteristics:
(a) The device response time is about ˜30 ms, which is faster than a graphene-based FET device (˜50-100 ms).
(b) The LOD is about 250 copies/ml, which is much lower than a graphene-based FET device (about 1000 copies/ml).
(c) The detection range is between 250 copies/ml to 2.5×10\hat{ }8 copies/ml, which is much larger than a graphene-based FET device (about 1×10\hat{ }3 copies/ml to 2.42×10\hat{ }7 copies/ml).

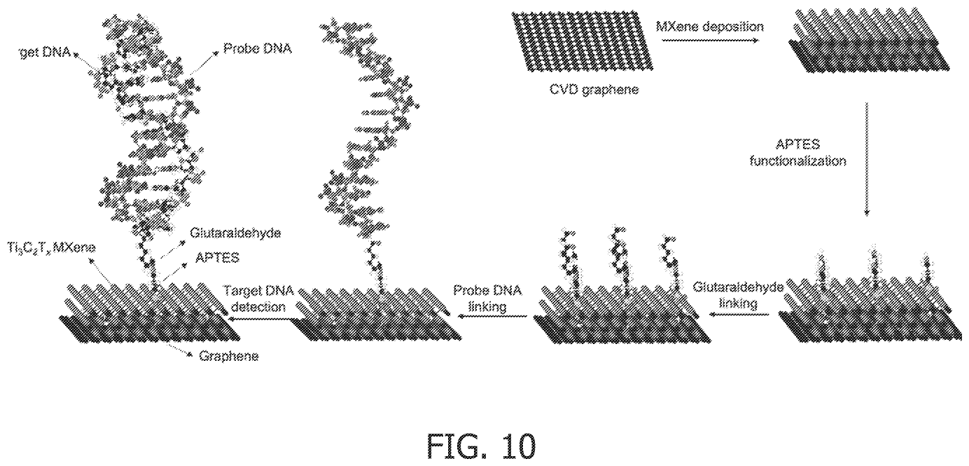

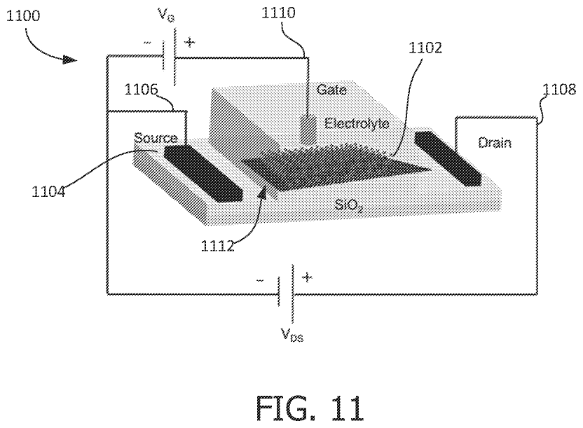

Aspects of the present disclosure also permit rapid detection of SARS-COV-2 and its Omicron variant viral nucleic acid using a high-efficiency MXene-graphene-based FET biosensor 1100. FIG. 10 illustrates a sensor functionalization process for nucleic acid detection according to this embodiment and FIG. 11 illustrates the MXene-graphene FET sensor 1100 for use with the process of FIG. 10 and configured to detect deoxyribonucleic acid (DNA) of the SARS-COV-2 Omicron variant.

As shown in FIG. 11, the FET sensor 1100 includes an ultra-sensitive VSTM 1102 on a substrate 1104, a source electrode 1106, a drain electrode 1108, and a gate 1110. The sensor 1100 combines the high chemical sensitivity of 2D transition metal carbide (MXene) and the continuity of large-area high quality graphene to form the VSTM 1102. Through polymer linking, sensor 1100 utilizes probe DNA linking to achieve electrochemical signal transduction when viruses are deposited onto the surface of VSTM 1102. In an embodiment, the MXene-graphene VSTM 1102 is configured to directly receive viruses. In an embodiment, sensor 1100 includes a microfluid receiver 1112, such as a channel or chamber, for collecting a sample for testing.

The biosensor 1100 comprises the integrated electrodes 1106, 1108 and, in an embodiment, 2D Ti₃C₂Tₓ MXene-graphene sensing material 1102. Referring to the functionalization process illustrated in FIG. 10, sensor 1100 further includes polymer, linker, and specialized probe DNA. The probe DNA contains complementary sequences that can recognize the nucleic acid of a target virus such as the Omicron variant. To functionalize VSTM 1102, the surface of MXene-graphene is modified with (3-Aminopropyl) triethoxysilane (APTES), followed by a glutaraldehyde treatment and probe DNA immobilization.

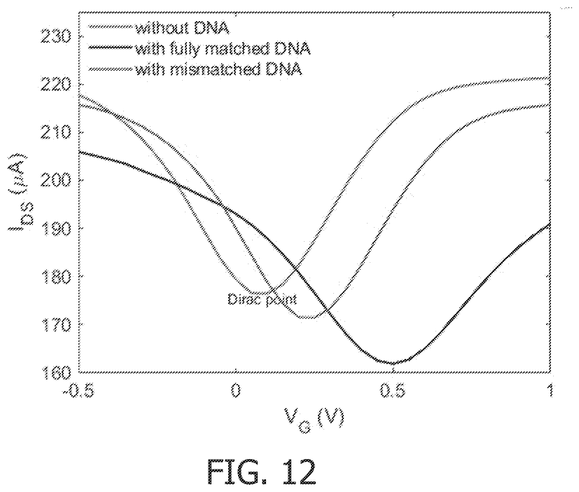

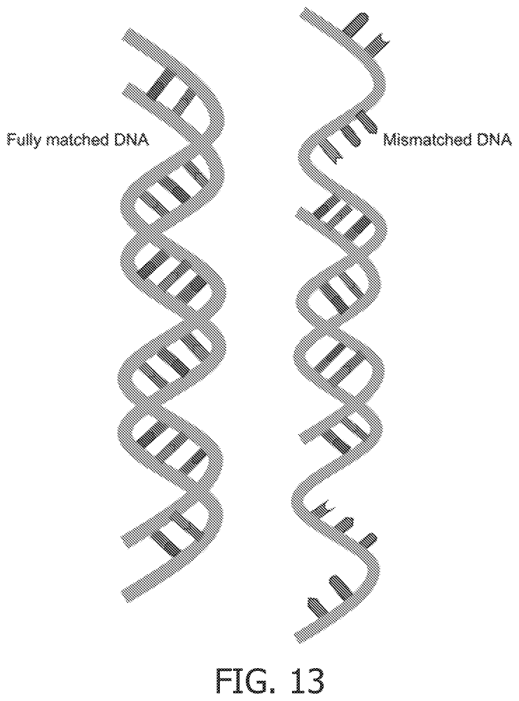

FIG. 12 provides example data for calibrating the sensor of FIG. 11, specifically, IDS versus V_{G} curves when probe DNA and target DNA fully match and mismatch. FIG. 13 illustrates fully matching and mismatching probe DNA and target DNA as detected using the sensor of FIG. 11. Although described in connection with the Omicron variant, it is to be understood that sensor 1100 is configurable for detecting DNA of other viruses.

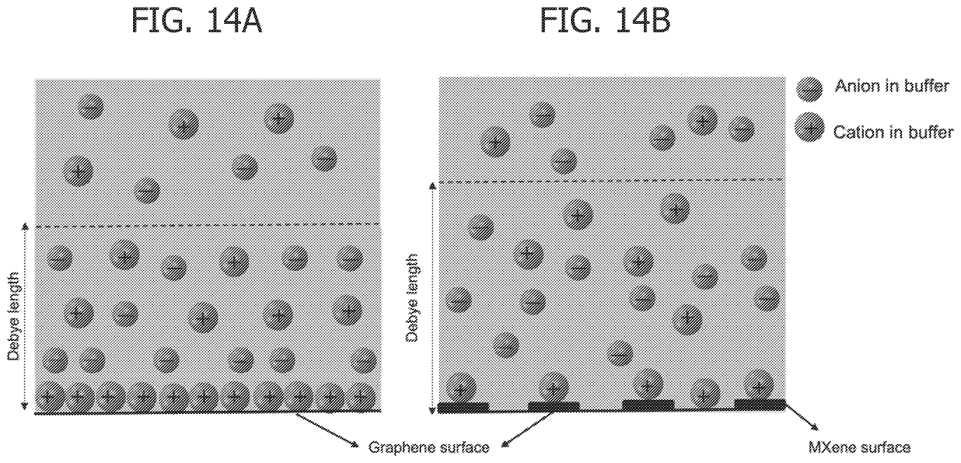

The example I_{DS} versus V_{G} curves shown in FIG. 12 display minimum conduction points, referred to as Dirac points. When the target DNA sequences fully match with probe DNA, the Dirac point shifting is much larger than for when the target DNA sequences partially match with probe DNA. Chemical vapor deposition (CVD) grown graphene is continuous and in wafer scale size, which makes it easy for FET fabrication. The equation for Dirac point shifting (ΔV_{D}) calculation is:

$$Δ {V}_{D} = \frac{e Δ n}{{C}_{T}}$$

where Δn is the change of charge carrier density of the sensing material, C_{T} is the capacitance between gate and the sensing material. After depositing MXene flakes onto graphene surface, the surface roughness increases, resulting in loosely structured electrical double layer (EDL) and the increasing of Debye length, resulting in the increase of Δn. FIGS. 14A and 14B compare EDL structures of graphene and MXene-graphene, respectively. In addition, higher surface roughness brings lower C_{T}. Therefore, using MXene-graphene composite as the sensing material significantly increases ΔV_{D} during FET sensing in comparison with that for using pure graphene film as the sensing material. This increased shifting provides lower limit of detection (LOD) for DNA sensing. For these reasons, sensor 1100 has a fast-response (e.g., within 30 seconds) and high sensitivity, and can sense specific properties.

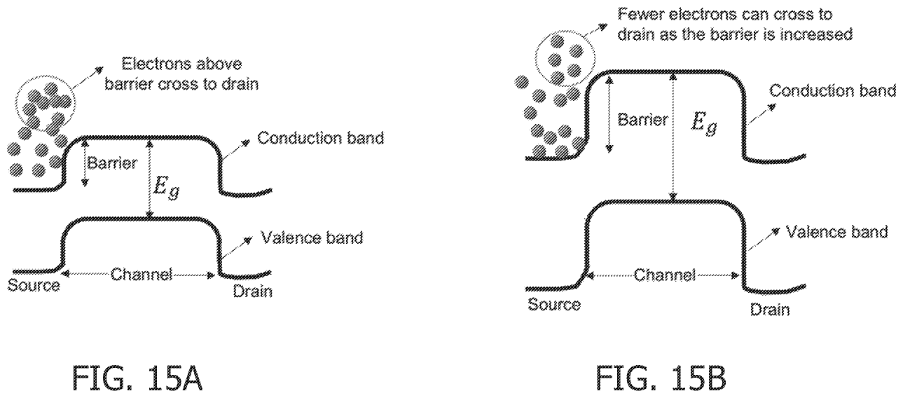

FIGS. 15A and 15B compare bandgap structures of graphene and MXene-graphene, respectively. Graphene has no band gap, as shown in FIG. 15A, which introduces low on/off current ratio when using graphene as the FET sensing material. As shown in FIG. 15B, MXene has band gap and after deposition of MXene onto graphene, MXene-graphene composite has band gap. The relationship between electronic mobility μ and band gap E_{g} is as follows:

$$μ ∝ {E}_{g}^{- \frac{3}{2}}$$

Advantageously, larger band gap introduces smaller electronic mobility, which results in larger Δn. Therefore, the deposition of MXene on graphene can significantly increase ΔV_{D}, which promotes MXene-graphene biosensor 1100 with higher sensitivity.

The order of execution or performance of the operations in accordance with aspects of the present disclosure illustrated and described herein is not essential, unless otherwise specified. That is, the operations may be performed in any order, unless otherwise specified, and embodiments may include additional or fewer operations than those disclosed herein. For example, it is contemplated that executing or performing a particular operation before, contemporaneously with, or after another operation is within the scope of the invention.

When introducing elements of the invention or embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively, or in addition, a component may be implemented by several components.

The above description illustrates embodiments by way of example and not by way of limitation. This description enables one skilled in the art to make and use aspects of the invention, and describes several embodiments, adaptations, variations, alternatives and uses of the aspects of the invention, including what is presently believed to be the best mode of carrying out the aspects of the invention. Additionally, it is to be understood that the aspects of the invention are not limited in its application to the details of construction and the arrangement of components set forth in the following description or illustrated in the drawings. The aspects of the invention are capable of other embodiments and of being practiced or carried out in various ways. Also, it will be understood that the phraseology and terminology used herein is for the purpose of description and should not be regarded as limiting.

It will be apparent that modifications and variations are possible without departing from the scope of the invention defined in the appended claims. As various changes could be made in the above constructions and methods without departing from the scope of the invention, it is intended that all matter contained in the above description and shown in the accompanying drawings shall be interpreted as illustrative and not in a limiting sense.

In view of the above, it will be seen that several advantages of the aspects of the invention are achieved and other advantageous results attained.

The Abstract and Summary are provided to help the reader quickly ascertain the nature of the technical disclosure. They are submitted with the understanding that they will not be used to interpret or limit the scope or meaning of the claims. The Summary is provided to introduce a selection of concepts in simplified form that are further described in the Detailed Description. The Summary is not intended to identify key features or essential features of the claimed subject matter, nor is it intended to be used as an aid in determining the claimed subject matter.

1. A sensor for detecting virus particles, comprising:
a substrate;
a field effect transistor (FET), the FET including source and drain electrodes formed on the substrate; and
a two-dimensional virus sensing transduction material (VSTM) film formed on the FET, the VSTM film configured to collect a sample collected from a subject, the VSTM film comprising MXene-graphene and having a probe corresponding to the virus particles to be detected linked thereto,
wherein a drain-source current response of the FET is representative of an amount of the virus particles in the sample.

2. The virus sensor set forth in claim 1, wherein electrodes of the FET and the VSTM film are printed directly on a surface of the substrate.

3. The virus sensor set forth in claim 1, wherein the virus particles to be detected are SARS-COV-2 virus particles and/or influenza A (H1N1).

4. The virus sensor set forth in claim 1, wherein the VSTM film comprises covalently bonded MXene-graphene hybrid continuous film.

5. The virus sensor set forth in claim 1, further comprising a microfluid receiver associated with the VSTM film configured to collect the sample from the subject.

6. The virus sensor set forth in claim 1, wherein the sample is collected from at least one of a bioaerosol, a solution, and a touch.

7. The virus sensor set forth in claim 1, wherein the VSTM film is functionalized for virus detection using an aminosilane for linking the corresponding probe.

8. The virus sensor set forth in claim 1, wherein the probe comprises an antibody corresponding to the virus particles to be detected and wherein the antibody linked to the VSTM film comprises influenza A (H1N1) antibody and/or SARS-COV-2 spike antibody.

9. The virus sensor set forth in claim 1, wherein the probe comprises a deoxyribonucleic acid (DNA) corresponding to the virus particles to be detected and wherein the DNA linked to the VSTM film comprises SARS-COV-2 DNA.

10. A method of fabricating a virus sensor comprising:
patterning a source electrode and a drain electrode of a field effect transistor (FET) on a substrate;
growing a monolayer graphene by chemical vapor deposition (CVD) and depositing the graphene on the substrate;
depositing a layer of MXene on the graphene to form a continuous virus sensing transduction material (VSTM) film formed on the FET, the VSTM film comprising MXene-graphene and configured to receive a sample from a subject for detecting particles of a virus; and
linking a probe corresponding to the virus to be detected to the VSTM film, wherein a drain-source current response of the FET is representative of an amount of particles of the particles of the virus in the sample.

11. The method set forth in claim 10, further comprising coating the patterned electrodes with a conductive silver coating.

12. The method set forth in claim 10, wherein the virus particles to be detected are SARS-COV-2 virus particles and/or influenza A (H1N1).

13. The method set forth in claim 10, wherein the VSTM film comprises covalently bonded MXene-graphene hybrid continuous film.

14. The method set forth in claim 10, further comprising forming a microfluid receiver associated with the VSTM film for collecting the sample from the subject and electrically connecting a gate of the FET to the microfluid receiver.

15. The method set forth in claim 10, wherein the sample is collected from at least one of a bioaerosol, a solution, and a touch.

16. The method set forth in claim 10, wherein linking the corresponding probe includes functionalizing the VSTM film for virus detection using an aminosilane.

17. The method set forth in claim 10, wherein the probe comprises an antibody corresponding to the virus particles to be detected and wherein the antibody linked to the VSTM film comprises influenza A (H1N1) antibody and/or SARS-COV-2 spike antibody.

18. The method set forth in claim 10, wherein the probe comprises a deoxyribonucleic acid (DNA) corresponding to the virus particles to be detected and wherein the DNA linked to the VSTM film comprises SARS-COV-2 DNA.

19. A face mask comprising:
a substrate configured to cover a subject's mouth and nose when worn by the subject; and
a sensor located on an inner surface of the substrate, the sensor configured to detect virus particles in exhaled breath of the subject when the substrate is worn by the subject, the sensor comprising:
a field effect transistor (FET), the FET including source and drain electrodes formed on the substrate; and
a two-dimensional virus sensing transduction material (VSTM) film formed on the FET, the VSTM film configured to collect a sample of the exhaled breath of the subject, the VSTM film comprising MXene-graphene and having a probe corresponding to the virus particles to be detected linked thereto,
wherein a drain-source current response of the FET is representative of an amount of the virus particles in the sample.

20. The face mask set forth in claim 19, wherein the sensor is printed directly on the inner surface of the substrate.

21. The face mask set forth in claim 19, wherein the sensor further comprises a microfluid receiver associated with the VSTM film configured to collect the sample from the subject.

22. The face mask set forth in claim 19, wherein the VSTM film is functionalized for virus detection using an aminosilane for linking the corresponding probe.

23. The face mask set forth in claim 19, wherein the probe comprises at least one of the following: influenza A (H1N1) antibody, SARS-COV-2 spike antibody, and SARS-COV-2 deoxyribonucleic acid (DNA).

24. The virus sensor set forth in claim 1, further comprising a resistance monitor system coupled to the FET for measuring the drain-source current response thereof representative of the amount of the virus particles in the sample.

25. The virus sensor set forth in claim 24, wherein the resistance monitor system comprises a detector circuit configured to measure one or both of drain-source a current signal and a drain-source voltage signal.

26. The virus sensor set forth in claim 25, wherein the detector circuit comprises an auto-balancing bridge impedance measurement circuit and a voltage divider resistance measurement circuit.

27. A sensor for detecting virus particles, comprising:
a substrate;
a resistive measuring electrode detector, the detector including two conducting electrodes formed on the substrate and separated from each other by a physical distance; and
a two-dimensional virus sensing transduction material (VSTM) film connecting the two conducting electrodes, the VSTM film configured to collect a sample from a subject, the VSTM film comprising MXene-graphene,
wherein a change in resistance measured between the two conducting electrodes by the resistive measuring electrode detector is representative of an amount of the virus particles in the sample.

28. The virus sensor set forth in claim 27, wherein the virus particles to be detected are SARS-COV-2 virus particles and/or influenza A.

29. The virus sensor set forth in claim 27, wherein the VSTM film comprises covalently bonded MXene-graphene hybrid continuous film.

30. The virus sensor set forth in claim 27, further comprising a microfluid receiver associated with the VSTM film configured to collect the sample from the subject.

31. The virus sensor set forth in claim 27, wherein the sample is collected from at least one of a bioaerosol, a solution, and a touch.

32. The virus sensor set forth in claim 27, wherein the VSTM film is functionalized for virus detection using an aminosilane for linking a probe corresponding to the virus particles to be detected thereto.

33. The virus sensor set forth in claim 32, wherein the probe comprises an antibody corresponding to the virus particles to be detected and wherein the antibody linked to the VSTM comprises influenza A antibody and/or SARS-COV-2 spike antibody.

34. The virus sensor set forth in claim 32, wherein the probe comprises a deoxyribonucleic acid (DNA) corresponding to the virus particles to be detected and wherein the DNA linked to the VSTM film comprises SARS-COV-2 DNA.

35. The virus sensor set forth in claim 27, wherein the resistive measuring electrode detector is configured to apply a voltage across the two conducting electrodes and to measure current in the VSTM film therebetween.

36. The virus sensor set forth in claim 27, wherein the resistive measuring electrode detector comprises an auto-balancing bridge impedance measurement circuit and a voltage divider resistance measurement circuit.

37. A method of fabricating a pathogen sensor comprising:
patterning two electrodes on a substrate, the electrodes separated from each other by a physical distance;
growing a monolayer graphene by chemical vapor deposition (CVD) and depositing the graphene on the substrate;
depositing a layer of MXene on the graphene to form a continuous virus sensing transduction material (VSTM) film formed between the electrodes, the VSTM film comprising MXene-graphene and configured to receive a sample from a subject for detecting particles of a pathogen; and
coupling a resistive measuring electrode detector to the electrodes, wherein a change in resistance between the electrodes measured by the resistive measuring electrode detector is representative of an amount of particles of the pathogen in the sample.

38. The method set forth in claim 37, further comprising coating the patterned electrodes with a conductive silver coating.

39. The method set forth in claim 37, wherein the pathogen particles to be detected are SARS-COV-2 virus particles and/or influenza A.

40. The method set forth in claim 37, wherein the VSTM film comprises covalently bonded MXene-graphene hybrid continuous film.

41. The method set forth in claim 37, further comprising forming a microfluid receiver associated with the VSTM film for collecting the sample from the subject.

42. The method set forth in claim 37, wherein the sample is collected from at least one of a bioaerosol, a solution, and a touch.

43. The method set forth in claim 37, further comprising functionalizing the VSTM film for virus detection using an aminosilane.

44. The method set forth in claim 37, further comprising linking an influenza A antibody and/or SARS-COV-2 spike antibody to the VSTM film.

45. The method set forth in claim 37, further comprising linking a deoxyribonucleic acid (DNA) corresponding to the pathogen particles to be detected to the VSTM film.

46. A sensor for detecting airborne virus particles, comprising:
a substrate configured to receive an airborne bioaerosol sample; and
a sensor located on the substrate, the sensor configured to detect virus particles in the sample, the sensor comprising:
a resistive measuring electrode detector, the detector including two conducting electrodes formed on the substrate and separated from each other by a physical distance; and
a two-dimensional virus sensing transduction material (VSTM) film connecting the two conducting electrodes, the VSTM film configured to collect a sample, the VSTM film comprising MXene-graphene,
wherein a change in resistance between the two conducting electrodes measured by the resistive measuring electrode detector is representative of an amount of the virus particles in the sample.

47. A method of detecting virus particles, comprising:
receiving a sample on a continuous virus sensing transduction material (VSTM) film formed between two conducting electrodes, the VSTM film comprising a layer of MXene deposited on a monolayer graphene;
coupling a resistive measuring electrode detector to the two conducting electrodes;
applying a voltage across the two conducting electrodes;
measuring a current between the two conducting electrodes by the resistive measuring electrode detector to determine a change in resistance measured therebetween before and after receiving the sample,
wherein the change in resistance between the two conducting electrodes measured by the resistive measuring electrode detector is representative of an amount of the virus particles in the sample.
